(19) United States
(12) Patent Application Publication
(10) Pub. No.: US 2026/0363963 A1
(43) Pub. Date: Nov. 19, 2026

(54) SCAN BODY FOR IMPLANTS, COMPRISING COATING LAYER WHICH REDUCES SURFACE REFLECTANCE AND INCREASES BIOCOMPATIBILITY

(71) Applicant: THE INDUSTRY & ACADEMIC COOPERATION IN CHUNGNAM NATIONAL UNIVERSITY (IAC), Daejeon (KR)

(72) Inventors: Sung Yun YANG, Daejeon (KR); In Young PARK, Daejeon (KR)

(21) Appl. No.: 18/997,438

(22) PCT Filed: Jul. 20, 2023
(86) PCT No.: PCT/KR2023/095033

§ 371 Date: Jan. 21, 2025

(30) Foreign Application Priority Data
Jul. 21, 2022 (KR) 10-2022-0089969

(51) Int. Cl. A61K 6/25; A61C 8/00; A61C 9/00; A61K 6/84; A61K 6/844; A61K 6/887; A61K 6/898

(52) U.S. Cl. CPC A61K 6/25; A61C 8/0001; A61C 9/0053; A61K 6/84; A61K 6/844; A61K 6/887; A61K 6/898

(57) ABSTRACT

Embodiments relate to an implant scan body or abutment including a coating layer with low surface reflectance and high biocompatibility, wherein the low surface reflectance makes it possible to obtain highly accurate scan data on the shape of a patient's tooth by preventing light reflection when an optical scanner is used to scan a tooth model, and the high biocompatibility prevents the occurrence of problems such as inflammation in mouth structures such as the gums.

### TECHNICAL FIELD

The present invention relates to an implant scan body including a coating layer with low surface reflectance and high biocompatibility.

### BACKGROUND ART

Implant treatment is a dental treatment method designed to mimic the function of a natural tooth by implanting a biocompatible implant body (fixture) into the jawbone whose volume has been increased to sufficiently cover the area where a tooth is missing or where a tooth has been extracted through additional surgical procedures such as bone grafting or bone expansion.

Implant treatment includes osseointegration, a morphological, physiological, and direct connection between the jawbone where normal function is maintained and the surface of the implant body, and then a process of bone remodeling of the jawbone around the implant. There are several types of implants, but recently, screw-shaped intraosseous implants have been mainly used.

The implant is generally composed of a fixture, an abutment, and a crown. The fixture is a part that acts as a root of the tooth and is mainly made of titanium. The abutment is a part that connects the crown to the fixture and corresponds to the neck of a natural tooth. The crown is the upper part of the implant, corresponds to the head of a natural tooth, and is made of gold or ceramic material depending on the position and condition of the tooth.

In implant treatment, first, the implant location is checked and a hole for accommodating the implant body is made in the jawbone by drilling. In other words, drilling is performed several times until the size matches the diameter of the implant body, and then the implant body (fixture) is carefully implanted. When satisfactory osseointegration of the implanted fixture is achieved, the process of connecting the abutment to the fixture and making an artificial tooth prosthesis (crown) is performed.

The artificial tooth prosthesis is made through a process of taking an impression of a tooth of the patient into whom the fixture has been implanted, creating a tooth model based on the impression, scanning the tooth model, and creating a prosthesis model that fits the shape of the patient's tooth based on the acquired scan data.

However, this method has a problem in that the process is complicated because it involves taking an impression and creating a model based on that impression. To overcome this problem, a method of inserting the abutment model directly into the patient's mouth and scanning the same may be considered.

In this case, in order to create a model of a prosthesis suitable for the shape of the patient's tooth, the scan data obtained by scanning the tooth model must include accurate fixture scan data (information on center coordinates and direction of the fixture screw hole).

However, when scan data of a tooth model are acquired using an optical scanner, there is an advantage in that it is possible to acquire scan data within a relatively short period of time, but there is a problem in that the scan data of the tooth model, acquired using the optical scanner, do not include accurate scan data of the fixture.

In addition, there is a problem in that, since the scan area is not fully exposed because it is covered by the gum after the abutment is attached to the fixture implanted into the patient's gum, it is difficult to obtain scan data of an abutment model that matches the shape of the patient's tooth.

Among the scanning methods, there are two types: a laser-based method and a photograph-based method. However, in the laser-based method, a scan body of a conventional abutment model made of a metal cannot be scanned accurately because it is sensitive to light reflection.

Accordingly, a method of reducing light reflection by spraying onto the abutment model may be considered, but there is a problem in that significant data errors may occur depending on the spraying method.

Accordingly, there is a need for the development of a coating composition that makes it possible to obtain highly accurate scan data on the shape of a patient's tooth by preventing light reflection on an abutment model, and that has high biocompatibility so that it does not cause adverse biological effects even when it forms a coating layer on the abutment model.

### PRIOR ART DOCUMENTS

### Patent Documents

(Patent Document 1) KR 10-1461105 B1

### DISCLOSURE

### Technical Problem

An object of the present invention is to provide an implant scan body including a coating layer with low surface reflectance and high biocompatibility.

Another object of the present invention is to provide a an implant scan body which has low surface reflectance, making it possible to obtain highly accurate scan data on the shape of a patient's tooth by preventing light reflection when an optical scanner is used to scan a tooth model, and which has high biocompatibility so that problems such as inflammation do not occur in mouth structures such as the gums.

Yet another object of the present invention is to provide an implant abutment including a coating layer with low surface reflectance and high biocompatibility.

### Technical Solution

To achieve the above objects, the present invention provides an implant scan body including a coating layer with low surface reflectance and high biocompatibility, including: an implant scan body made of a metal material; and a coating layer formed on the surface of the scan body, wherein the coating layer may include a hyaluronic acid (HA) or alginic acid coating layer, a poly(allylamine hydrochloride) (PAH) coating layer, and a CYD coating layer including a compound represented by Formula 1 below:

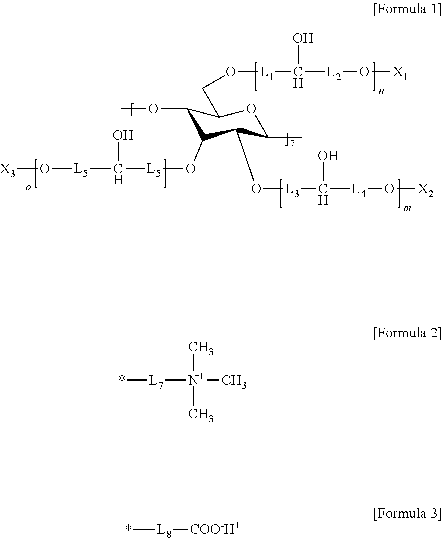

wherein
* denotes a bonding site;
n, m and o are the same as or different from one another and are each independently an integer ranging from 1 to 100;
L₁ to L₈ are the same as or different from one another and are each independently a single bond or a substituted or unsubstituted alkylene group having 1 to 10 carbon atoms; and
X₁ to X₃ are the same as or different from one another and are each independently selected from the group consisting of cyclodextrin, the compound represented by Formula 2 above, and the compound represented by Formula 3 above.

The coating layer may include: a first coating layer including a compound selected from the group consisting of the hyaluronic acid (HA) or alginic acid coating layer, the poly(allylamine hydrochloride) (PAH) coating layer, and a mixture thereof; and a CYD coating layer formed on the surface of the first coating layer.

The first coating layer may be one in which the hyaluronic acid or alginic acid coating layer and the poly(allylamine hydrochloride) coating layer are alternately formed.

The compound represented by Formula 1 in the CYD coating layer may be a cationic polymer (Cat-CyD) including the compound represented by Formula 2.

The metal material may be zirconia, titanium, chrome, cobalt, platinum, gold, or an alloy thereof.

An implant abutment according to another embodiment of the present invention includes: an implant abutment made of a metal material; and a coating layer formed on a surface of the abutment, wherein the coating layer may include a hyaluronic acid (HA) or alginic acid coating layer, a poly(allylamine hydrochloride) (PAH) coating layer, and a CYD coating layer including a compound represented by Formula 1 below:

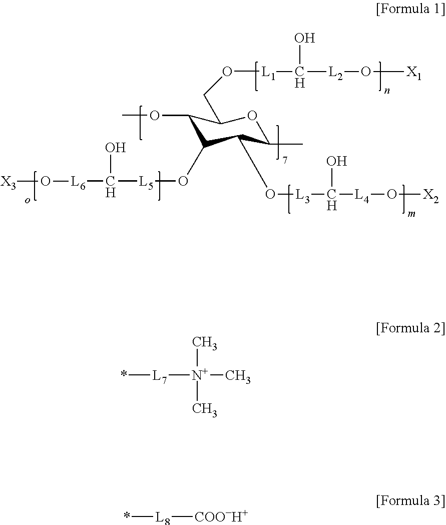

wherein
* denotes a bonding site;
n, m and o are the same as or different from one another and are each independently an integer ranging from 1 to 100;
L₁ to L₈ are the same as or different from one another and are each independently a single bond or a substituted or unsubstituted alkylene group having 1 to 10 carbon atoms; and
X₁ to X₃ are the same as or different from one another and are each independently selected from the group consisting of cyclodextrin, the compound represented by Formula 2 above, and the compound represented by Formula 3 above.

### Advantageous Effects

The present invention relates to a scan body or abutment having a coating layer formed thereon, which has low surface reflectance, making it possible to obtain highly accurate scan data on the shape of a patient's tooth by preventing light reflection when an optical scanner is used to scan a tooth model, and which has high biocompatibility so that problems such as inflammation do not occur in mouth structures such as the gums.

### BRIEF DESCRIPTION OF DRAWINGS

FIG. 1 shows the results of measuring the reflectance of a titanium surface having a coating layer formed thereon according to one Example of the present invention.

FIG. 2 shows the results of MTT assay according to one Example of the present invention.

FIG. 3 shows the results of an experiment on the anti-inflammatory effect of a silicone material having a coating layer formed thereon according to one Example of the present invention.

FIG. 4 shows the results of a 3D printing experiment using a coating composition according to one Example of the present invention.

### BEST MODE

The present invention relates to an implant scan body including a coating layer with low surface reflectance and high biocompatibility, including: an implant scan body made of a metal material; and a coating layer formed on the surface of the scan body, wherein the coating layer may include a hyaluronic acid (HA) or alginic acid coating layer, a poly(allylamine hydrochloride) (PAH) coating layer, and a CYD coating layer including a compound represented by Formula 1 below:

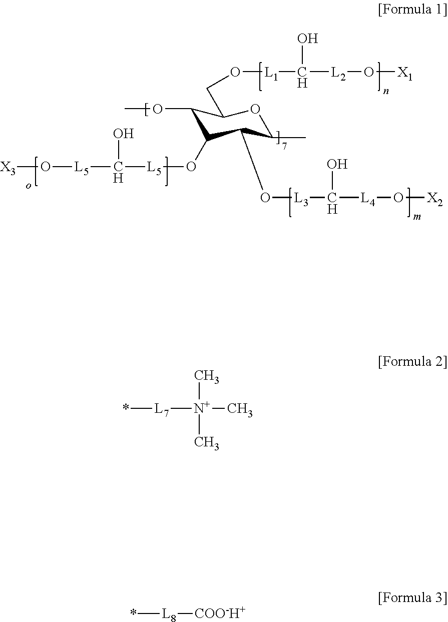

wherein * denotes a bonding site; n, m and o are the same as or different from one another and are each independently an integer ranging from 1 to 100; L₁ to L₈ are the same as or different from one another and are each independently a single bond or a substituted or unsubstituted alkylene group having 1 to 10 carbon atoms; and X₁ to X₃ are the same as or different from one another and are each independently selected from the group consisting of cyclodextrin, the compound represented by Formula 2 above, and the compound represented by Formula 3 above.

### MODE FOR INVENTION

Hereinafter, embodiments of the present invention will be described in detail so that those skilled in the art to which the present invention pertains can easily carry out the present invention. However, the present invention may be embodied in various different forms and is not limited to the embodiments described herein.

In the present invention, hyaluronic acid is meant to include pure hyaluronic acid (HA) or a salt thereof, or include both the pure hyaluronic acid and salt thereof.

In the present invention, alginic acid is meant to include pure alginic acid or a salt thereof, or both the pure alginic acid and salt thereof. The salt of alginic acid may be, for example, sodium alginate (SA).

The implant of the present invention refers to a medical device for in vivo implantation that is implanted in vivo to perform its intended function, and the dental implant is meant to include a fixture that is implanted in or on the jawbone (implanted between the gum and the gum bone) for the purpose of supporting a prosthesis for restoring a missing tooth.

The implant is generally composed of a fixture, an abutment, and a crown. The implant of the present invention refers to a concept including a fixture, an abutment, and a crown.

An implant abutment according to one embodiment of the present invention is an implant abutment for connecting the crown to the fixture, wherein the abutment is made of a metal material, and the abutment made of the metal material may have a coating layer formed thereon that prevents light reflection during scanning and has excellent biocompatibility.

A procedure for implanting the implant will be briefly described below.

First, the site of implantation of a fixture is drilled using a drill. Thereafter, the fixture is implanted into the alveolar bone in the drilled area and osseointegrated to form an artificial tooth root.

After a certain period of time, when osseointegration of the fixture is completed, an abutment is inserted into and fitted with the fixture, and then fastened with an abutment screw so that the fixture and the abutment become integrated with each other.

Then, the abutment is covered with a crown, that is, a final artificial tooth prosthesis having the same shape as a natural tooth, and the implant procedure may be completed.

If the gum needs to be shaped to allow for proper healing before the artificial tooth prosthesis is placed, a healing abutment may be pre-attached to the fixture. In this case, the healing abutment may be used temporarily until the artificial tooth is placed.

In order to obtain a three-dimensional shape using a digital oral scanner and make a prosthesis based thereon, a three-dimensional image of the inside of the mouth is obtained using the digital oral scanner. In this case, a scan body is fixed to a fixture and then scanning is performed. When scanning is performed to obtain a three-dimensional image of the inside of the mouth as described above, the purpose of using a separate scan body is to accurately design the abutment fixed to the fixture as well as the crown by accurately identifying the exact location and direction of the hexagonal groove formed inside the fixture.

That is, a separate component called a scan body is used to recognize the thickness of the gums, which differs depending on the patient, the resulting implantation depth, and the exact direction.

As a method to obtain an image of the inside of the mouth, the above-mentioned scan body may be used, or a scan abutment may be used.

This artificial tooth prosthesis is made through a process of taking an impression of a patient's tooth, creating a model in a dental laboratory based on the impression, then combining an abutment model as an implant scan abutment with the model, scanning the same, and then matching the CAD data of the implant scan abutment to the scan data in a CAD program to create a prosthesis model that fits the shape of the patient's tooth.

However, the above-described scan body and abutment are generally made of metal material, and thus there is a problem in that the metal material reflects light, making it difficult to accurately scan the shape of the patient's tooth.

Generally, the metal material used to make the scan body and the abutment may be zirconia, titanium, chromium, cobalt, platinum, gold, or an alloy thereof, with titanium being preferred, but is not limited to the above examples.

To solve the above-described problem, a method of forming a coating layer on the surface of the metal material to prevent light reflection may be used. The coating layer is usually formed using a compound, but as described below, problems may arise in that the coating layer may cause inflammation and also has low adhesion to titanium.

That is, a problem may arise in that the coating layer is not uniformly formed on the surface of titanium or the coating layer easily detaches.

Due to the above problem, if it is not easy to form a titanium coating layer, a problem arises in that the effect of preventing light reflection by the coating layer is relatively low.

In order to prevent the above problem, the present invention is characterized by forming a coating layer on the surface of a metal material. In particular, the coating layer has excellent adhesion to titanium and reduces light reflection, making it possible to acquire an accurate image of the inside of the mouth using the digital oral scanner.

Specifically, the coating layer may include a hyaluronic acid or alginic acid coating layer, a poly(allylamine hydrochloride) (PAH) coating layer, and a CYD coating layer including a compound represented by Formula 1 below:

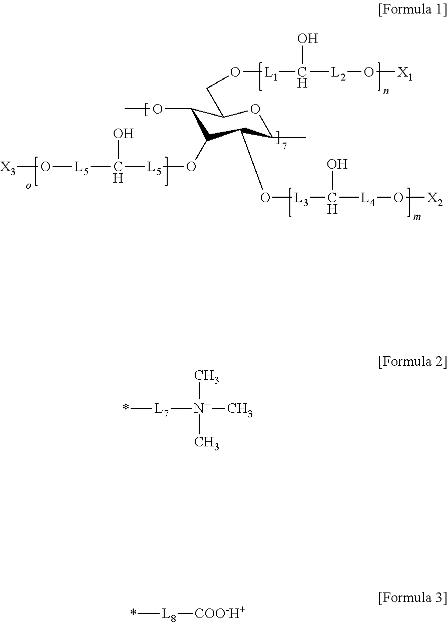

wherein
* denotes a bonding site;
n, m and o are the same as or different from one another and are each independently an integer ranging from 1 to 100;
L₁ to L₈ are the same as or different from one another and are each independently a single bond or a substituted or unsubstituted alkylene group having 1 to 10 carbon atoms; and
X₁ to X₃ are the same as or different from one another and are each independently selected from the group consisting of cyclodextrin, the compound represented by Formula 2 above, and the compound represented by Formula 3 above.

The hyaluronic acid (HA) polymer is a compound represented by Formula 4 below:

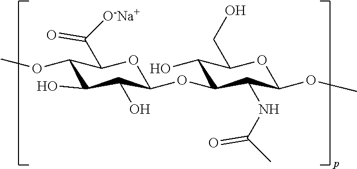

wherein
p is an integer ranging from 1 to 100.

The alginic acid is a compound represented by Formula 5 below:

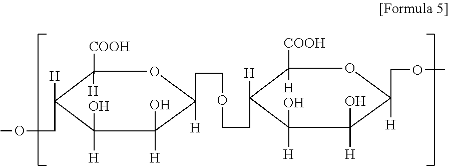

The poly(allylamine hydrochloride) (PAH) is a compound represented by Formula 6 below:

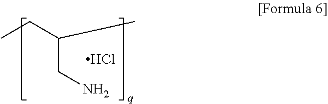

wherein
q is an integer ranging from 1 to 100.

Specifically, the coating layer formed on the surface of the scan body or the abutment may include: a first coating layer including a compound selected from the group consisting of the hyaluronic acid (HA) coating layer, the poly(allylamine hydrochloride) (PAH) coating layer, and a mixture thereof; and the CYD coating layer formed on the surface of the first coating layer.

PAH forming the first coating layer may be a cationic polymer compound, and HA may be an anionic polymer compound.

When the compound represented by Formula 1 may be a cationic polymer compound (Cat-CyD) when the quaternary ammonium salt represented by Formula 2 is bonded thereto, and may be an anionic polymer compound (Ani-CyD) when the compound represented by Formula 3 is bonded thereto.

The first coating layer may include a bilayer as one repeating unit, wherein the bilayer may include the above-described cationic polymer compound and anionic polymer compound as one set.

The first coating layer may be formed by depositing a plurality of [PAH/HA] layers. Here, the [PAH/HA] means that poly(allylamine hydrochloride) and the hyaluronic acid polymer are deposited as a single layer.

After the first coating layer is formed, a CYD coating layer may be formed on the surface of the first coating layer.

In addition, a method for forming a coating layer according to another embodiment of the present invention may include steps of: alternately depositing a hyaluronic acid (HA) polymer and poly(allylamine hydrochloride) (PAH) to form a first coating layer; and depositing a polymer represented by Formula 1 below to form a CYD coating layer:

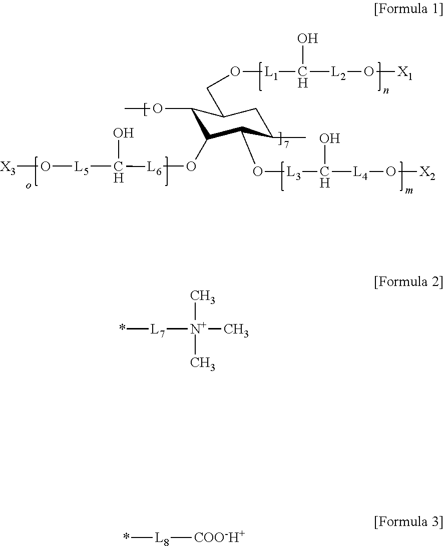

wherein
* denotes a bonding site;
n, m and o are the same as or different from one another and are each independently an integer ranging from 1 to 100;
L₁ to L₈ are the same as or different from one another and are each independently a single bond or a substituted or unsubstituted alkylene group having 1 to 10 carbon atoms; and
X₁ to X₃ are the same as or different from one another and are each independently selected from the group consisting of cyclodextrin, the compound represented by Formula 2 above, and the compound represented by Formula 3 above.

The compound represented by Formula 1 in the CYD coating layer may be a cationic polymer (Cat-CyD) including the compound represented by Formula 2.

### Preparation Example 1

### Production of Cationic Cat-CyD Polymer

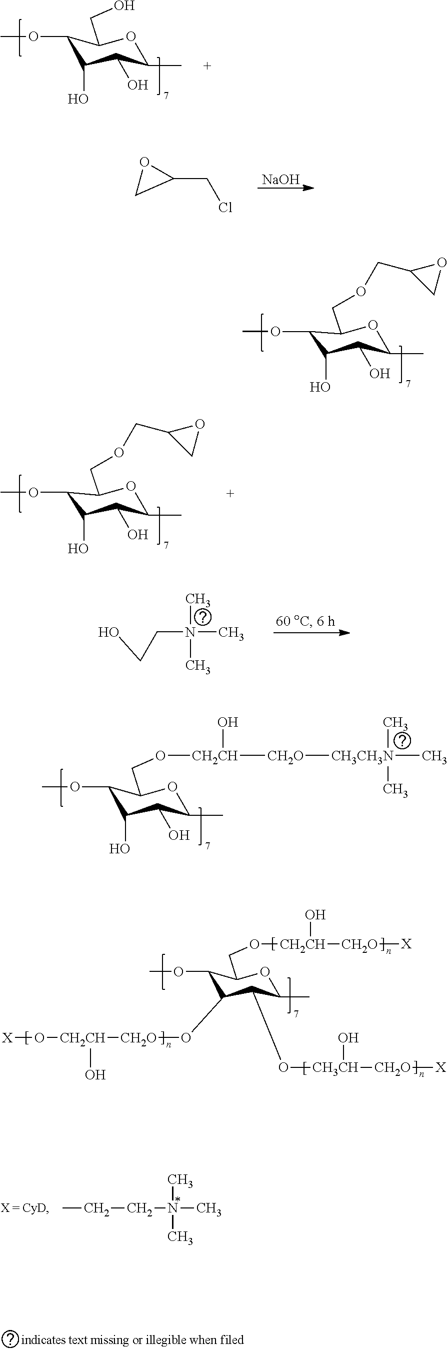

1 g (0.025 mol) of NaOH was dissolved in 20 ml of DI water in a 50 ml round-bottom flask, thereby preparing an aqueous solution containing 0.022 mol % (0.048 wt %) of NaOH. Then, 1.135 g (0.001 mol) of β-CyD was added thereto, followed by stirring at 25 °C for 18 hours. After completion of stirring, the round-bottom flask containing the reaction product was transferred into an oil bath and the hot plate temperature was set to 60 °C. When the set temperature was reached, 1.388 g (1.2 ml, 0.015 mol) of epichlorohydrin (EP) was added thereto at a rate of about 0.1 ml/min, and then immediately 0.2792 g (0.002 mol) of choline chloride (CC) was quickly added to the reactor which was then closed with a joint stopper. While the inside of the reactor was maintained at 60 °C during polymerization, and the mixture was allowed to react for 24 hours. Next, the pH of the reaction product was adjusted to neutral using an aqueous solution of 12N HCl and the polymerization was terminated. For purification of the obtained solution, dialysis was performed for 48 hours using MWCO 1000 and 3500 membranes depending on the intended use. The resulting solution was subjected to a freeze-drying process to form pure powder.

Yield: 0.74 g (22.2%)

### Preparation Example 2

### Formation of Coating Layer on Titanium Surface

A hyaluronic acid (HA) polymer and poly(allylamine hydrochloride) (PAH) were purchased and used. The Cat-CyD polymer was synthesized in the same manner as in Preparation Example 1.

Each polymer electrolyte was adjusted to a concentration of 0.01 M using double-distilled water and maintained at a pH of 3.0 or 4.5. A washed and dried titanium substrate was immersed in the polymer electrolyte solution to deposit the polymer on the surface.

Specifically, the titanium substrate was first immersed in a cationic PAH solution for 15 minutes to induce PAH adsorption, and then washed twice with double-distilled water to remove residual PAH. In the same manner as introducing the PAH, anionic HA was introduced with PAH to form a [PAH/HA] coating layer. The immersion and recovery were repeated to form a deposited layer with a desired thickness. Thereafter, the resulting structure was immersed in a cationic Cat-β-CyD solution to form a coating layer.

### Preparation Example 3

### Formation of Silicone Coating Layer on Silicone Surface

A coating layer was formed on a silicone material in the same manner as in Preparation Example 2.

### Experimental Example 1

### Results of Reflectance Measurement

After the coating layer was formed on the titanium substrate as described in the Preparation Example above, the degree of reduction in reflectance was evaluated through experiments.

As comparative examples, uncoated titanium (bare Ti foil), titanium having a [CYD/HA]₆ coating layer formed thereon, and titanium having a [CYD/HA]_{6.5} coating layer formed thereon were also evaluated.

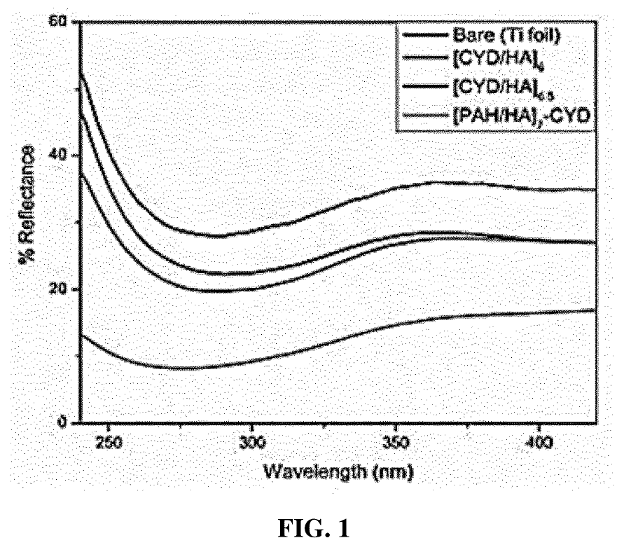

The experimental results are shown in FIG. 1.

It was confirmed that the titanium having the coating layer formed thereon in Preparation Example 2 showed a reduction in reflectance of 50% or more compared to the uncoated titanium, indicating that the coating layer had an excellent effect of preventing light reflection.

### Experimental Example 2

### Evaluation of In Vivo Toxicity

To analyze cell viability, trypan blue cell counting assay and MTT assay were performed using each appropriately selected substrate.

For cell counting, cells grown on each substrate for a specific period of time were detached from the substrate using trypsin-EDTA, and stained with trypan blue to distinguish between live cells and dead cells, live cells were counted using a hemocytometer, and calculation was performed using the following equation.

$$Total Viable cells / ml = \frac{number of live cells \times {10}^{4} cells / ml}{number of squares counted} \times {}^{⋆} Dilution Factor$$

$${}^{⋆} Dilution Factor = \frac{Volume of cell suspension + volume of Trypan blue}{Volume of cell suspension}$$

For MTT assay, the cells were treated with 5 mg/ml of thiazolyl blue tetrazolium bromide (MTT; ab146345, Abcam) per well, incubated for 2 hours in an incubator at 37 °C under 5% CO₂, and then dissolved using dimethyl sulfoxide (DMSO). Next, the absorbance was measured at 570 nm.

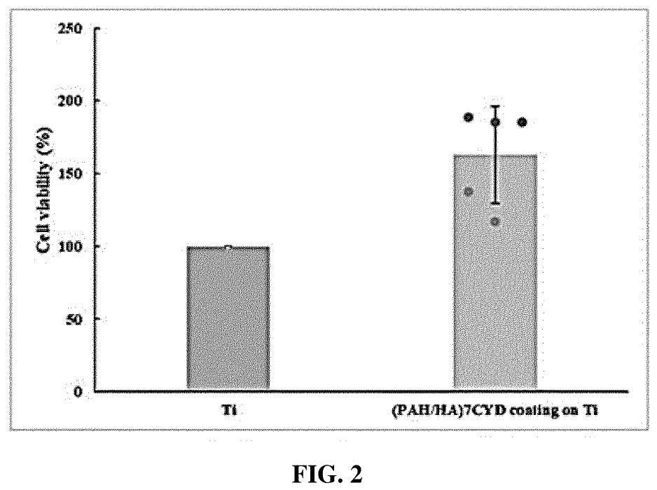

The experimental results are shown in FIG. 2. The cell viability increased by 160% or more when the cells were incubated on the (PAH/HA)₇-CYD-coated sample compared to when the cells were incubated on the uncoated titanium (Ti) surface. Through the above experiment, it can be seen that the scan body or abutment having the coating layer formed thereon according to the present invention may be used as a product with reduced light reflectance and no cytotoxicity, compared to a scan body or abutment made of a bare titanium material.

### Experimental Example 3

### Inflammation Inhibitory Effect

The silicone having the coating layer formed thereon in Preparation Example 3 was implanted into an experimental mouse.

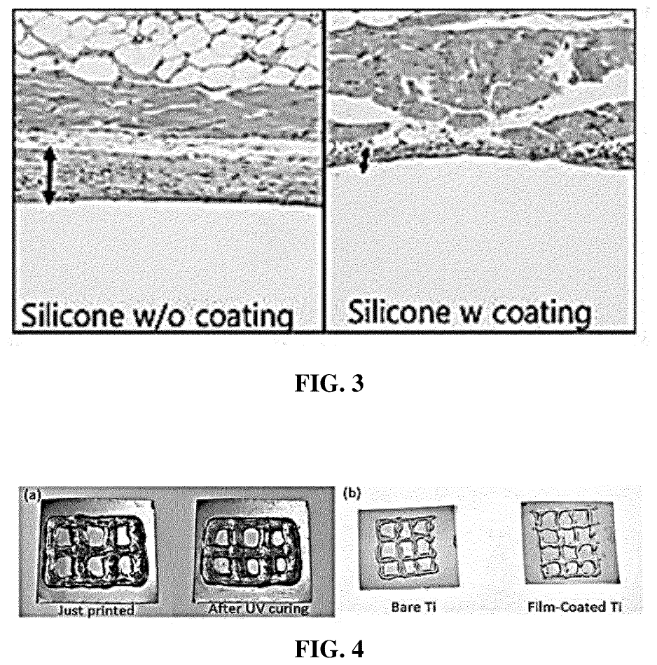

The experimental results are shown in FIG. 3.

Referring to FIG. 3, as a result of observation 30 days after implantation into the experimental mouse, it was confirmed that the area where the surrounding tissue was encapsulated due to side effects was significantly reduced.

### Experimental Example 4

### 3D (Stereoscopic) Printing Experiment

As an ink to be printed, a gel was prepared by mixing hyaluronic acid or sodium alginate, cationic Cat-β-CyD, and a UV-reactive cross-linker (benzophenone, it is not necessary to limit the cross-linker to this one compound, so it is necessary to decide whether to list it, or simply refer to it as a cross-linker, or list several related compounds) at a weight ratio of 2:1:1 (w/w/w). The gel was placed into a printing nozzle, printed on a titanium foil (Ti-foil) substrate coated with a reflection-reducing coating, and subjected to a cross-linking reaction using a UV (254 nm) lamp.

The experimental results are shown in FIG. 4.

The result in the left of FIG. 4(a) shows the printed state of the composite ink and shows that the printed shape spread without being maintained over a long period of time, and the result in the right shows that the printed shape was well maintained and fixed by UV cross-linking. FIG. 4(b) shows a comparison between the case where the ink was printed on the bare Ti foil substrate and then cross-linked, and the case where the ink was printed and cross-linked on the Ti substrate surface having the [PAH/HA]m+ [CYD/SA]n coating film, and it was confirmed that, in the case where the coating film was formed, the ink spreading phenomenon was reduced due to a faster cross-linking reaction.

Although the preferred embodiments of the present invention have been described in detail above, the scope of the present invention is not limited thereto, and various modifications and improvements can be made by those skilled in the art without departing from the basic concept of the present invention as defined in the following claims and also fall within the scope of rights of the present invention.

### INDUSTRIAL APPLICABILITY

The present invention relates to an implant scan body including a coating layer with low surface reflectance and high biocompatibility.

1. An implant scan body comprising a coating layer with low surface reflectance and high biocompatibility, comprising:
an implant scan body made of a metal material; and
a coating layer formed on a surface of the scan body,
wherein the coating layer comprises a hyaluronic acid (HA) or alginic acid coating layer, a poly(allylamine hydrochloride) (PAH) coating layer, and a CYD coating layer comprising a compound represented by Formula 1 below: 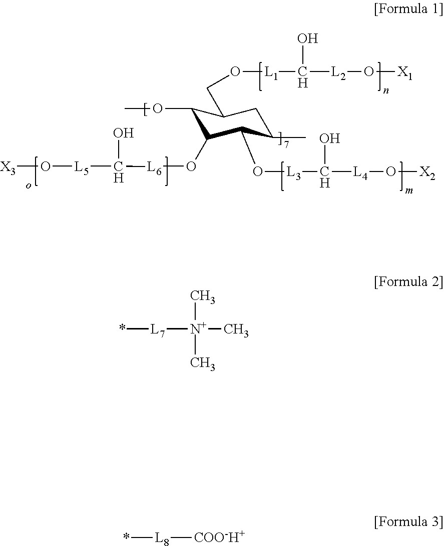
wherein
* denotes a bonding site;
n, m and o are the same as or different from one another and are each independently an integer ranging from 1 to 100;
L₁ to L₈ are the same as or different from one another and are each independently a single bond or a substituted or unsubstituted alkylene group having 1 to 10 carbon atoms; and
X₁ to X₃ are the same as or different from one another and are each independently selected from the group consisting of cyclodextrin, a compound represented by Formula 2 above, and a compound represented by Formula 3 above.

2. The implant scan body of claim 1, wherein the coating layer comprises:
a first coating layer comprising a compound selected from the group consisting of the hyaluronic acid (HA) coating layer, the poly(allylamine hydrochloride) (PAH) coating layer, and a mixture thereof; and
the CYD coating layer formed on a surface of the first coating layer.

3. The implant scan body of claim 2, wherein the first coating layer is one in which the hyaluronic acid coating layer and the poly(allylamine hydrochloride) coating layer are alternately formed.

4. The implant scan body of claim 2, wherein the compound represented by Formula 1 in the CYD coating layer is a cationic polymer (Cat-CyD) comprising the compound represented by Formula 2.

5. The implant scan body of claim 1, wherein the metal material is zirconia, titanium, chrome, cobalt, platinum, gold, or an alloy thereof.

6. An implant abutment comprising a coating layer with low surface reflectance and high biocompatibility, comprising:
an implant abutment made of a metal material; and
a coating layer formed on a surface of the abutment,
wherein the coating layer comprises a hyaluronic acid (HA) coating layer, a poly(allylamine hydrochloride) (PAH) coating layer, and a CYD coating layer comprising a compound represented by Formula 1 below: 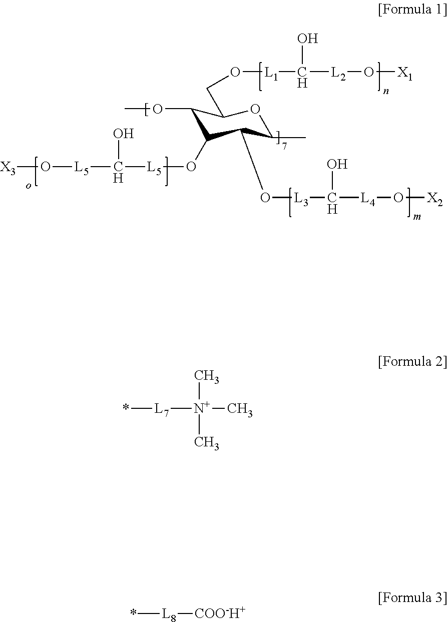
wherein
* denotes a bonding site;
n, m and o are the same as or different from one another and are each independently an integer ranging from 1 to 100;
L₁ to L₈ are the same as or different from one another and are each independently a single bond or a substituted or unsubstituted alkylene group having 1 to 10 carbon atoms; and
X₁ to X₃ are the same as or different from one another and are each independently selected from the group consisting of cyclodextrin, a compound represented by Formula 2 above, and a compound represented by Formula 3 above.
